(19) United States
(12) Patent Application Publication
(10) Pub. No.: US 2012/0330191 A1
(43) Pub. Date: Dec. 27, 2012

(54) Intramedullary Rod Tracking

(76) Inventors: Urs HULLIGER, Langendorf (CH); Adrian BAUMGARTNER, Langendorf (CH)

(21) Appl. No.: 13/227,007

(22) Filed: Sep. 7, 2011

Related U.S. Application Data
(60) Provisional application No. 61/380,871, filed on Sep. 8, 2010.

(51) Int. Cl. A61B 5/103

(52) U.S. Cl. 600/587

(57) ABSTRACT

A device for determining deformation of an intramedullary rod comprises a longitudinal probe sized and shaped to be inserted into a channel of the rod and including a deflection detection element, a head portion including a lumen extending therethrough and a coupling element to attach the head portion to a proximal end of the intramedullary rod, the lumen slidably receiving the probe therein, and a logging unit configured to detect relative movement between the probe and the head portion and to determine a current path of the intramedullary rod from the relative movement.

### PRIORITY CLAIM

The present application claims priority to U.S. Provisional Application Ser. No. 61/380,871 entitled “Intramedullary Rod Tracking” filed on Sep. 8, 2010 to Urs Hulliger and Adrian Baumgartner. The entire contents of this application are incorporated herein by reference thereto.

### FIELD OF THE INVENTION

The present invention relates to the field of intramedullary rod tracking and, more particularly, to a system and a method for tracking an intramedullary rod and detecting deformation of the rod with regard to the curvature of the rod as it is inserted into an object, such as a bone, and more particularly, into a medullary cavity of a bone.

### BACKGROUND

The use of intramedullary nails or rods is well known and an established form of treatment of fractures in long bones, especially, for example, in the femur, tibia or humerus. The intramedullary rod may be inserted into a medullary cavity of a bone to treat bone fractures in orthopedic surgery and intramedullary tumors occurring in some forms of cancer. The rod includes at least one fixing hole arranged at the distal end and/or the proximal end thereof to aid in attachment thereof to the bone. The fixing hole may have an internal thread for mating with a cross lock or screw inserted in the fixing hole through the bone. A targeting device including an aiming arm may be used outside of the bone to determine where a transverse hole should be drilled to insert the cross lock or screw through the fixing hole. In some cases, however, the rod may bend during insertion of the rod into the bone such that the aiming arm must be adjusted to take the bending of the rod into account.

There is accordingly a need in the art for improved methods and systems as well as devices that at least partially overcome the above-mentioned problems. Specifically, there is a need in the art for an improved system and method with regard to intramedullary rod tracking which is easy to handle when determining a possible deformation of an intramedullary rod, and wherein manufacturing the device is uncomplicated and the components used therein are simple.

### SUMMARY OF THE INVENTION

The present invention relates to a tracking system for an intramedullary rod and a method for tracking an intramedullary rod being inserted in a medullary canal. The present invention also relates to an improved tracking system including simple built-up components.

The present invention further relates to a system and a method for obtaining a three-dimensional view of the intramedullary rod being inserted into the medullary cavity with minimal experience of the operator.

The present invention also relates to a method which compares a deviation of a curvature between the intramedullary rod before and after implantation into a bone. The difference of these measurements determines the deviation, which is further used to aid in aligning an aiming device with a bore extending through the intramedullary nail.

In accordance with an exemplary embodiment of the present invention, a tracking system for an intramedullary rod comprises a probe insertable into the rod and movable toward a distal end of the rod. The probe further includes a deflection detection element. The probe is slidably receivable within a head portion attachable to a proximal end of the intramedullary rod. The system further comprises a logging unit adapted to detect relative movement between the probe and the head portion. By inserting the probe into the rod, the relative movement between the probe and the head portion is measured. The logging unit uses the relative movement to determine the depth of the probe within the cavity and to obtain the orientation of the probe (rotation) with regard to the three-dimensional information of a bending obtained based on the determined deflection. Thus, a three-dimensional view of the rod inserted in the medullary cavity may be obtained. The probe may also be rotated relative to the rod. It may also be possible to measure an amount of deflection as the probe is removed from the rod to verify the accuracy of the information obtained by inserting the probe into the rod.

In an exemplary embodiment, the logging unit recognizes at least one parameter from the relative movement and uses the at least one parameter to determine the current path of the intramedullary rod. The parameter or combination of parameters is selected from the group consisting of an amount of rotation of the probe with regard to a reference orientation between the head and the probe; an amount of deflection of the probe with regard to a reference orientation; and an amount of insertion of the probe relative to the head portion. The deflection detection element may be adapted to detect a deflection of the probe while the logging unit is configured to simultaneously collect the amount of deflection detected, the depth of insertion of the probe and the amount of rotation thereof.

The logging unit may also be configured to determine the current path of the intramedullary rod by comparing the relative movement against an expected movement of the probe. The expected movement may be predetermined by inserting the probe into the intramedullary rod before the rod is inserted into, for example, a patient.

In another exemplary embodiment, the deflection detection element may be one of a laser/mirror triangulation device, a tension stripe, a strain gauge strip, and/or a magnetic sensor, the deflection detection element being formed of simple components which are easy to handle during manufacturing. The above-identified deflection detection devices have a high degree of reliability and an appropriate resolution and accuracy for measuring the deflection of the probe. Any of the above-mentioned elements of the probe may be easily connected to the logging unit through the stem via an electrical connection such as, for example, a wire.

In another exemplary embodiment, the above-identified deflection detection element may be arranged at least partially inside the probe such that the deflection detection element is protected against an inner wall of the intramedullary rod. The laser/mirror triangulation device may be constructed using an interferometry measurement method as disclosed in U.S. Pat. No. 5,541,731 and U.S. Pat. No. 5,751,588 for general construction purposes.

In another exemplary embodiment, the logging unit may be a microprocessor having re-writable memory for storing data corresponding to the parameters recognized from the relative movement. The parameters may be one or more of an amount of deflection measured; an amount of insertion; and an amount of rotation for current procedure and/or for a plurality of past procedures.

In addition to the deflection detection element, the probe may also include a sensor for determining a position of a locking hole in the intramedullary rod. Thus, two different sensors may be used for obtaining information with regard to the three-dimensional imaging of the intramedullary rod and the detection of the locking hole. Having a lock hole detection sensor in addition to the deflection detection element ensures that the respective results are obtained independently from each other to provide reliability and redundancy.

In a further exemplary embodiment, the probe may comprise pads arranged along an outer surface thereof for centering the probe in the intramedullary rod. The pads may be preferably arranged in such a way that the probe is centered in the cavity of the intramedullary rod over its entire length. This will help obtain a reliable measurement of the deflection and also protect the probe against the inner surface of the intramedullary rod. The pads may have a triangular shape extending including a point extending radially outward relative to the longitudinal axis of the probe, which in one exemplary embodiment, may be in the shape of a cylinder.

In a further embodiment, the system may include a control unit connectable to the logging unit. The control unit may be adapted to calculate a difference between a curved trajectory of the probe in a deformed intramedullary rod and a straight and/or non-deformed intramedullary rod using the data collected by the logging unit. The control unit has a display on which the calculated difference may be displayed in terms of a number with digits and a sign. This display preferably provides the results of the measurements in an efficient manner such that the displayed number may be entered in the aiming device for the correction of the alignment of the aiming arm such that a hole may be drilled at the correct position in the bone for a bone fixation element to be inserted through the bone into the locking hole of the intramedullary rod.

In another exemplary embodiment of the present invention a method for tracking an intramedullary rod being inserted in a medullary cavity of a bone comprises inserting a probe attached to a probe stem into the rod, and locating and fixing a head at a proximal end of the rod, wherein the probe stem is slidably arranged with respect to the head. Further, the method comprises moving the probe in the rod, and while moving the probe in the rod in the direction of the proximal end of the rod, measuring relative movement between the probe and the head portion. The relative movement measure may be one or a combination of: the deflection of the probe; the distance the probe has been inserted in the rod; and/or the orientation of the probe with respect to a reference orientation. The data may be associated with each other, i.e. the deflection of the rod, the distance the probe has been inserted into the rod and the orientation of the probe with regard to a reference orientation, are collected. In an exemplary embodiment, for each point in time at which data is collected, there will be the information about distance, deflection and orientation. The method provides an easy to use intuitive procedure for obtaining tracking information about the intramedullary rod.

In an exemplary embodiment, the deflection of the probe may be measured by any sensor device capable of detecting deflection or bending of the probe. In particular, the deflection of the probe may be measured using one of a laser/mirror triangulation device, a tension stripe, a strain gauge strip, and a magnetic sensor.

In another exemplary embodiment, the deflection of the probe may also be measured as the probe is moved proximally toward a proximal end of the rod to verify the previously measured values. Thus, the necessary removal of the probe from the rod may provide additional information, minimizing the time and/or interaction of the probe with the intramedullary rod.

According to a further exemplary embodiment of the invention a method for tracking insertion of an intramedullary rod with a channel into a medullary cavity of a bone comprises measuring the intramedullary rod before implantation into the medullary cavity thereby generating reference data. Further, the method comprises measuring the intramedullary rod after implantation into the medullary cavity and calculating correction value(s) for adjustment of an aiming device to aligning the aiming device with the actual position of locking hole(s) in the intramedullary rod after insertion.

The present invention is directed to a device for determining deformation of an intramedullary rod comprising a longitudinal probe sized and shaped to be inserted into a channel of the rod and including a deflection detection element and a stem coupled to the probe, a head portion including a lumen extending therethrough and a coupling element to attach the head portion to a proximal end of the intramedullary rod. The lumen slidably receives the stem such that the probe is movable relative to the head portion and motion of the probe relative to the head portion corresponds to motion of the probe relative to the intramedullary rod. A logging unit coupled to the head portion detects a distance of travel of the probe relative to the head portion, a rotation of the probe relative to the channel and a deflection of the probe, the distance of insertion detected by measuring a relative movement of the probe with respect to the head portion, the rotation of the probe detected by measuring an amount of rotation of the probe with regard to a reference orientation between the head and the probe, the deflection detected by measuring of deflection of the deflection detection element with respect to the reference orientation. The logging unit aggregates data corresponding to the amount of deflection, the distance of travel of the probe and the amount of rotation along the path of travel of the probe to determine a current path of the intramedullary rod.

In another exemplary embodiment, an aiming device is attached to an intramedullary rod before implantation of the rod into the medullary cavity so that the aiming device may be calibrated to the particular intramedullary rod with which it is to be used. This overcomes problems relating to, for example, manufacturing tolerances which may cause certain features of the rod to deviate from their target positions.

### BRIEF DESCRIPTION OF THE DRAWINGS

A further understanding of the nature and advantages of the present invention may be realized by reference to the remaining portions of the specification and the drawings wherein the same reference numerals are used throughout the several drawings to refer to similar components.

FIG. 1 is a schematic view of an exemplary embodiment of a tracking system for an intramedullary rod according to the present invention;

FIG. 2 is a schematic view showing the tracking system of FIG. 1 being inserted in an intramedullary rod;

FIG. 3 is a schematic view of a further exemplary embodiment of a tracking system for an intramedullary rod according to the present invention;

FIG. 4a shows an enlarged schematic view of a tracking system including an alternate deflection detection element according to the present invention;

FIG. 4b show in an enlarged schematic view an exemplary embodiment of a tracking system according to the present invention elucidating a determination of bending of an inserted intramedullary rod; and

FIG. 5a shows a schematic view of a head portion of the tracking system of FIG. 1, attached to a proximal end of an intramedullary rod that is inserted into a medullary cavity of a bone according to the present invention;

FIG. 5b shows a schematic view of a probe of the tracking system of FIG. 1 moved relative to the intramedullary rod; and

FIG. 5c shows a schematic view of a probe of the tracking system of FIG. 1, detecting a position of a locking hole of the intramedullary rod.

### DETAILED DESCRIPTION

The present invention may be further understood with reference to the following description and the appended drawings, wherein like elements are referred to with the same reference numerals. The present invention relates to the treatment of bone fractures and, in particular, relates to devices for determining deflection of intramedullary rods during insertion into a bone. It will be understood by those of skill in the art that an intramedullary rod may bend as it is inserted into a medullary canal of a bone, causing a deflection along a length thereof. Exemplary embodiments of the present invention describe a tracking device including a probe that may be inserted into a channel of an intramedullary rod to measure deflection thereof and to determine the position of one or more locking holes extending through the deflected intramedullary rod. It will be understood by those of skill in the art that the measured deflection of the intramedullary rod and/or the position of the locking holes may be used to adjust an aiming device such that an opening of the aiming device aligns with the actual position of the locking hole(s) in the deflected intramedullary rod. It should be noted that the terms “proximal” and “distal” as used herein, refer to a direction towards (proximal) and away from (distal) a surgeon or other user of the device.

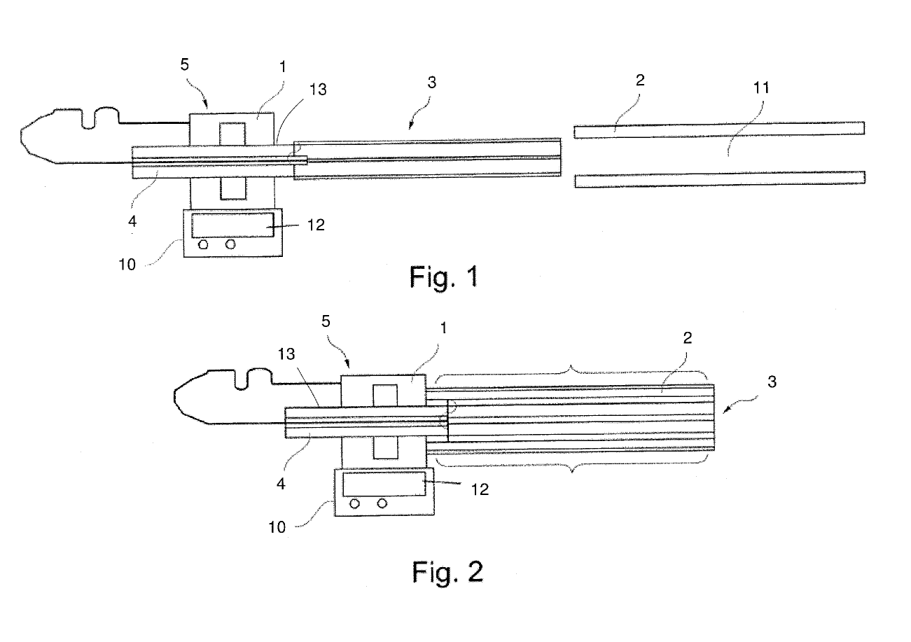

As shown in FIGS. 1-3, a device 1 according to an exemplary embodiment of the invention comprises a longitudinal probe 3 for insertion into an intramedullary rod 2 and a head portion 13 that connects the device 1 to the intramedullary rod 2. The probe 3 and the head portion 13 are slidable relative to one another via a stem portion 4. The device 1 further comprises a logging unit 5 detecting data corresponding to a depth of insertion, an orientation and a deflection of the probe 3 and a control unit 10 that calculates the deflection of the intramedullary rod 2 using the data collected by the logging unit 5. The intramedullary rod 2 extends from a proximal end to a distal end and includes a channel 11 extending from the proximal end through at least a portion thereof. The intramedullary rod 2 further includes at least one locking hole 7 extending transversely therethrough.

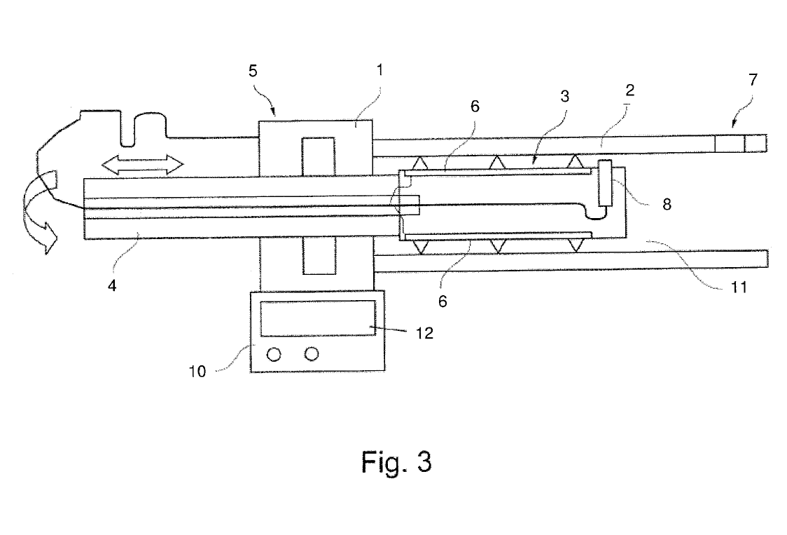

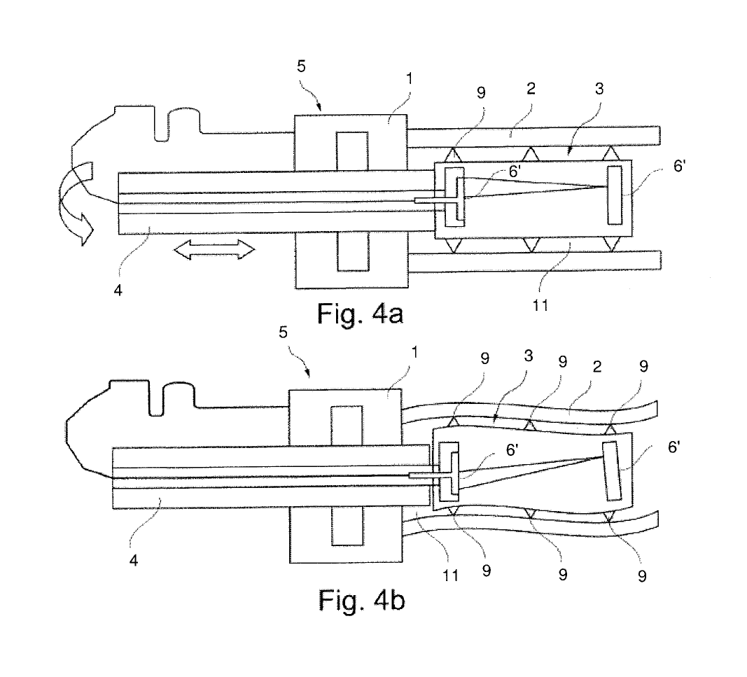

As shown in FIG. 3, the probe 3 is a longitudinal element sized and shaped for insertion into the channel 11 of the rod 2. The probe 3 may include a plurality of pads 9 mounted on an outer surface thereof for centering the probe 3 within the channel 11. The pads 9 may be substantially triangular including a pointed end extending radially outward from the probe 3 to contact an inner surface of the channel 11. It will be understood by those of skill in the art, however, that the pads 9 may take any of a variety of shapes and sizes so long as the pads 9 facilitate centering of the probe 3 within the channel 11. The probe 3 also includes a deflection detection element 6 and a sensor 8 for detecting the locking hole 7 of the intramedullary rod 2. The deflection detection element 6 may extend within the probe 3 along at least a portion of a length of the probe 3 and detects a deflection of the probe 3 as it is inserted into the channel 11. The deflection detection element 6 may be, for example, a tension stripe, a strain gauge or a magnetic sensor. The sensor 8 may be positioned at a distal end of the probe 3 and may detect the locking hole 7 by rotating and or moving the probe 3 longitudinally relative to the intramedullary rod 2. In an alternate embodiment, as shown in FIGS. 4a and 4b, a deflection detection element 6′ may be a laser/mirror triangulation device.

The stem portion 4 may also be substantially longitudinal, extending proximally from a proximal end of the probe 3. The stem portion 4 is sized and shaped to be slidable within the head portion 13. Thus, an outer circumference of the stem 4 may be slightly smaller than an inner circumference of the head portion 13. The inner circumference of the head portion 13 is smaller than an outer circumference of the probe 3 such that the probe 3 cannot be slid within the head portion 13. The head portion 13 provides a sliding support for the probe stem 4. The head portion 13 may also include a coupling element for coupling the head portion 13 to a proximal end of the intramedullary rod 2 such that the head portion 13 remains stationary relative to the intramedullary rod 2 while the probe 3 is movable relative thereto. The head portion 13 may also include a movable element (not shown) configured to measure longitudinal movement of the probe 3 relative to the head portion 13. The movable element may be, for example, a wheel that turns in a first direction (e.g., clockwise) as the probe 3 is moved distally relative to the head portion 13 and an intramedullary rod 2 to which it is coupled and a second direction (e.g., counter clockwise) as the probe 3 is moved proximally relative to the head portion 13. It will be understood by those of skill in the art, however, that the movable element may be any element capable of detecting longitudinal movement of the probe 3 relative to the head portion 13 and the intramedullary rod 2 to which it is coupled.

The logging unit 5 may be connected to the head portion 13 through a wired or wireless connection and includes a data handling device for detecting and collecting information regarding an amount/distance of insertion of the probe 3 into the channel 11 of the rod 2, an orientation of the of the probe 3 relative to the intramedullary rod 2 and a bend or curvature of the path of the probe 3. The logging unit 5 may detect the amount or distance of insertion of the probe 3 in the channel 11 of the intramedullary rod 2 by measuring a relative longitudinal movement of the probe 3 with respect to the head portion 13. For example, where the movable element is a wheel, the logging unit 5 may detect the number of turns in the first direction to determine a length or distance of insertion of the probe 3 distally into the channel 11. Thus, the length or distance of insertion of the probe 3 into the rod 2 may be measured automatically by the logging unit 5. The logging unit 5 may also be capable of detecting an orientation of the probe 3 relative to the channel 11 of the rod 2 by measuring a rotation of the probe 3 relative to the head portion 13. Thus, the orientation of the probe 3 in the rod 2 may also be measured automatically by the logging unit 5. The logging unit 5 may measure the length of insertion and the orientation of the probe 3 simultaneously.

Further, the logging unit 5 may detect a deflection or curvature of the probe 3 measured by the deflection detection element 6 which may be connected to the logging unit 5 via a wire or cable passing through the stem 4. Since the probe 3 is sized and shaped for insertion into the channel 11 of the intramedullary rod 2, a deflection or bending of the probe 3 corresponds to a bending of the rod 2 caused by insertion of the rod 2 in the medullary cavity of the bone. This is schematically shown in FIG. 4a and FIG. 4b.

The logging unit 5 collects and stores the length, orientation and deflection data. The logging unit 5 may be, for example, a microprocessor having re-writable memory for storing the data. The data measured at the same point in time are associated with one another as a single data reference. Thus, each data reference may contain information regarding a specific time and point along the rod 2 and includes three measurements—the length, orientation and deflection. Moving the probe 3 within the channel 11 of the rod 2 will permit the logging unit 5 to obtain information for several points along the rod 2 so that a map of the rod 2 may be created. Thus, a three-dimensional image of the rod 2 may also be obtained so a sufficient number of points along the rod 2 are measured.

To determine the position of the locking hole 7, which may extend transversely through the intramedullary rod 2, the probe 3 may be rotated and moved within the channel 11 until the sensor 8 indicates alignment with the locking hole 7. While the probe 3 is rotated and/or moved longitudinally within the channel 11, the data regarding the length of insertion, orientation and deflection of the probe 3, and thereby the intramedullary rod 2, is collected.

The control unit 10 may be connected to the logging unit 5. The control unit 10 is a data processing arrangement which calculates a difference between the trajectory of the probe 3 in the actual intramedullary rod 2 and the trajectory the probe would have shown when inserted into the intramedullary rod 2 before it was deformed (e.g., prior to insertion into the medullary cavity of the bone). The difference is calculated using the data references collected by the logging unit 5. For convenience, the control unit 10 may further comprises a display 12 on which the calculated difference is displayed in terms of a number with digits. The display 12 may also display a sign indicating an objective assessment of the differential or corresponding directly to an adjustment which should be made to the aiming device to obtain a desired alignment of the aiming device with corresponding features of the intramedullary rod 2 (e.g., a locking hole 7). In either case, a user of the device 1 may use the data displayed to achieve the desired alignment of the aiming device with the intramedullary rod 2.

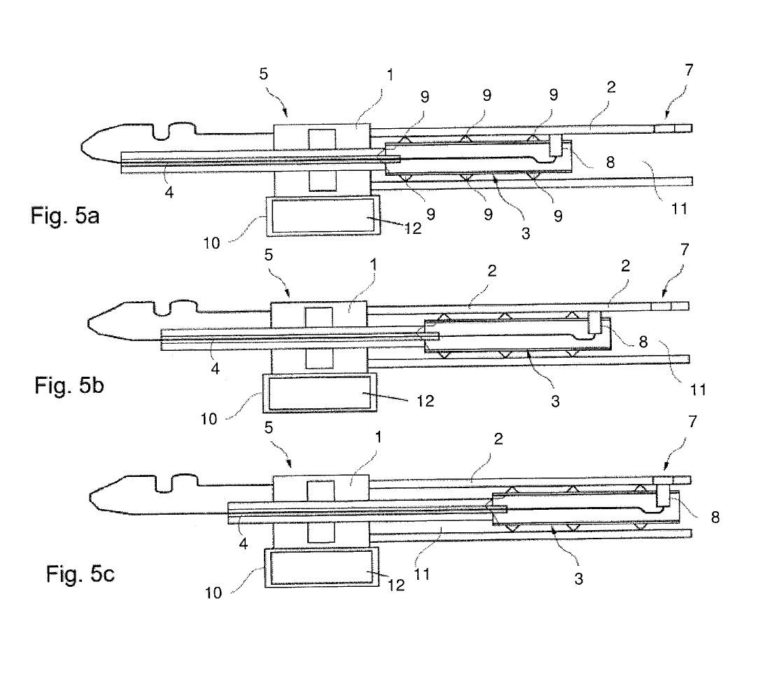

FIGS. 5a-5c show an exemplary method for tracking the intramedullary rod 2 after it has been inserted in a medullary cavity of a bone. As shown in FIG. 5a, the probe 3 is inserted into the channel 11 of the rod 2 until the head portion 13 comes into coupling contact with the proximal end of the intramedullary rod 2. The stem 4 is slidable relative to the probe 3 and the head portion 13 such that the probe 3 may be moved relative to the head portion 13. As shown in FIG. 5b, the probe 3 may be moved within the channel 11 of the rod 2 by pushing, pulling and/or rotating the probe stem 4. As the probe 3 is moved toward the distal end of the rod 2, the device 1 measures the deflection of the probe 3, the depth of insertion of the probe 3 within the channel 11 and the orientation of the probe 3 with regard to a reference orientation. The measured data is collected by the logging unit 5. Simultaneously measured data is categorized together such that all three measurements from a given time relate to one point of information along the rod 2.

Data collected by the logging unit 5 as the probe 3 is moved within the channel 11 may be used to calculate a deformity of the intramedullary rod 2 and subsequently displayed on the control unit 10 to aid in determining a corresponding adjustment that should be made to an aiming device (not shown) for drilling a hole into the bone in alignment with the locking hole 7. As shown in FIG. 5c, the probe 3 is moved within the channel 11 until the sensor 8 detects the position of the fixing hole 7.

Although the exemplary embodiments of the present invention and some of its advantages have been described in detail, it should be understood that various changes, substitutions, and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the present invention, processes, machines, manufacture, composition of matter, means, methods, or steps, presently existing or later to be developed that perform substantially the same function or achieve substantially the same result as the corresponding embodiments described herein may be utilized according to the present invention.

It will be appreciated by those skilled in the art that various modifications and alterations of the invention can be made without departing from the broad scope of the appended claims. Some of these have been discussed above and others will be apparent to those skilled in the art.

What is claimed is:

1. A device for determining a deformation of an intramedullary rod including a channel extending along a longitudinal axis thereof, comprising:
a longitudinal probe sized and shaped to be inserted into the channel of the rod and including a deflection detection element;
a head portion including a lumen extending therethrough and a coupling element arranged to attach the head portion to a proximal end of the intramedullary rod, the lumen configured to slidably receive the probe therein; and
a logging unit configured to detect relative movement between the probe and the head portion and to determine a current path of the intramedullary rod based on the determined relative movement.

2. The device according to claim 1, wherein the logging unit recognizes at least one parameter based on the determined relative movement and uses the at least one parameter to determine the current path of the intramedullary rod.

3. The device according to claim 2, wherein the logging unit is configured to determine the current path of the intramedullary rod based on one of a distance of travel of the probe relative to the head portion, a rotation of the probe relative to the channel, and a deflection of the probe, wherein, when the current path of the rod is determined based on the distance of travel of the probe, the distance of travel of the probe is detected by measuring movement of the probe relative to the head portion, and, when the current path of the rod is determined based on the rotation of the probe, the rotation of the probe is detected by measuring an amount of rotation of the probe with regard to a reference orientation between the head and the probe and, when the current path of the rod is determined based on the deflection of the probe, the deflection of the probe is detected by measuring a deflection of the deflection detection element with respect to the reference orientation.

4. The device according to claim 3, wherein the reference orientation corresponds to one of (a) an axis of the probe, (b) a central axis of the channel, and (c) a previously measured curve of the intramedullary rod in a non-deformed configuration.

5. The device according to claim 3, wherein the logging unit is configured to determine the current path of the intramedullary rod by comparing relative moment against an expected movement of the probe, the probe being movable relative to the head portion and motion of the probe relative to the head portion corresponds to motion of the probe relative to the intramedullary rod.

6. The device according to claim 1, wherein the logging unit is coupled to the head portion.

7. The device according to claim 1, wherein the deflection detection element includes one of a laser/mirror triangulation device, a tension stripe, a strain gauge strip and a magnetic sensor, the deflection detection element being arranged at least partially inside the probe.

8. The device according to claim 1, wherein the logging unit includes a microprocessor and a re-writable memory for storing data including the relative movement, the memory being arranged to store at least one of the parameters recognized from the relative movement, the parameters selected from an amount of deflection of the probe, a distance of travel of the probe, and an amount of rotation of the probe.

9. The device according to claim 1, wherein the probe includes a sensor for determining a position of a locking hole extending transversely through the intramedullary rod.

10. The device according to claim 1, wherein the probe includes a plurality of pads along an outer surface thereof for centering the probe in the channel of the intramedullary rod.

11. The device according to claim 1, further comprising a control unit connected to the logging unit, the control unit calculating a difference between a trajectory of the probe in the intramedullary rod after insertion into a target bone and a projected trajectory of the probe in the intramedullary rod in a non-deformed configuration, the device including a display on which the calculated difference is displayed.

12. The device according to claim 1, furthering comprising a stem coupled to the probe.

13. A method for tracking a path of an intramedullary rod after insertion in a target bone comprising:
a) inserting a device into a channel of an intramedullary rod, the device including a probe, a head portion and a stem movably coupling the probe to the head portion, the probe including a deflection detection element;
b) attaching the head portion to a proximal end of the intramedullary rod;
c) moving the probe relative to the head portion and the channel of the intramedullary rod; and
d) measuring relative movement between the probe and the head portion.

14. The method according to claim 13, wherein in step d) the relative movement measured includes at least one of: a distance of travel of probe from the head portion into the rod; a rotation of the probe relative to a reference orientation; and a deflection of the probe relative to the reference orientation.

15. The method according to claim 13, wherein the deflection detection element of the probe includes one of a laser/mirror triangulation device, a tension stripe, a strain gauge strip, and a magnetic sensor.

16. The method according to claim 14, wherein the distance, the rotation and the deflection of the probe with regard to the reference orientation are measured as the probe is moved distally into the rod and as the probe is withdrawn proximally from the rod.

17. The method according to claim 13, further comprising measuring the intramedullary rod prior to implantation in the medullary cavity to generate a reference path of the rod in a non-deformed state and the reference orientation.

18. The method according to claim 13, further comprising detecting a position of a locking hole extending transversely through the intramedullary rod.

19. The method according to claim 18, further comprising calculating, based on at least one of the measured distance, deflection and rotation of the probe, a correction value for adjusting an aiming device such that an opening thereof is aligned with the locking hole of the intramedullary rod.

20. The method according to claim 14, further comprising collecting data associated with each of the distance, rotation and deflection measurements.
